(19) United States
(12) Patent Application Publication
(10) Pub. No.: US 2011/0033893 A1
(43) Pub. Date: Feb. 10, 2011

(54) IMPROVED METHODS FOR PROTEIN PRODUCTION

(76) Inventors: Robert L. Garcea, Boulder, CO (US); Anton Middelberg, Brookfield (AU); Yap Pang Chuan, St. Lucia (AU)

Correspondence Address: SHERIDAN ROSS PC, 1560 BROADWAY, SUITE 1200, DENVER, CO, 80202, US

(21) Appl. No.: 12/669,997

(22) PCT Filed: Aug. 27, 2008
(86) PCT No.: PCT/US08/74483

§ 371 Date: Jul. 21, 2010

Related U.S. Application Data
(60) Provisional application No. 60/968,216, filed on Aug. 27, 2007.

(51) Int. Cl. C12P 21/00

(52) U.S. Cl. 435/69.7; 435/69.1

(57) ABSTRACT

Methods for low cell-density bacterial protein expression that can achieve levels of up to 180 mg/l using a simple and low cost strategy. Full codon optimization is unnecessary to improve expression of viral genes rich in E. coli rare codons. Using a strategically modified host cell provides a simpler and cheaper alternative.

### TECHNICAL FIELD

The invention relates to efficient and inexpensive protein production methods.

### BACKGROUND OF INVENTION

The cloning and expression of foreign proteins and polypeptides in bacteria such as Escherichia coli has revolutionized the study of protein function. The ability to produce and purify specific proteins in microbial hosts has also provided a wealth of diagnostic and therapeutic agents in recent years. One example is the production of capsid proteins from human papillomavirus (HPV) strains for use as diagnostic agents and therapeutic vaccines.

Papillomaviruses infect a wide variety of different species of animals including humans. Infection is typically characterized by the induction of benign epithelial and fibro-epithelial tumors, or warts at the site of infection. Each species of vertebrate is infected by a distinct group of papillomavirus, each papillomavirus group comprising several different papillomavirus types. For example, more than sixty different human papillomavirus genotypes have been isolated. Papillomaviruses are highly species-specific infective agents. For example, canine and rabbit papillomaviruses cannot induce papillomas in heterologous species such as humans. Neutralizing immunity to infection against one papillomavirus type generally does not confer immunity against another type, even when the types infect a homologous species.

In humans, papillomaviruses cause genital warts, a prevalent sexually transmitted disease. HPV types 6 and 11 are most commonly associated with benign genital warts condyloma acuminata. Genital warts are very common and subclinical or inapparent HPV infection is even more common than clinical infection. While most HPV induced lesions are benign, lesions arising from certain papillomavirus types, e.g., HPV-16 and HPV-18, can undergo malignant progression. Moreover, infection by one of the malignancy associated papillomavirus types is considered to be a significant risk factor in the development of cervical cancer, the second most common cancer in women worldwide. Of the HPV genotypes involved in cervical cancer, HPV-16 is the most common, being found in about 50% of cervical cancers.

In view of the significant health risks posed by papillomavirus infection generally, and human papillomavirus infection in particular, various groups have reported the development of recombinant papillomavirus antigens and their use as diagnostic agents and as prophylactic vaccines. In general, such research has been focused toward producing prophylactic vaccines containing the major capsid protein (L1) alone or in combination with the minor capsid protein (L2). For example, Ghim et al, Virology, 190:548-552 (1992) reported the expression of HPV-1 L1 protein using a vaccinia expression in Cos cells which displayed conformational epitopes and the use thereof as a vaccine or for serological typing or detection. In addition, the production of HPV-6 and HPV-11 L1 protein and HPV-6, HPV-11, HPV-16 and HPV-18 virus-like particles using a baculovirus/insect cell expression system (WO 94/20137) and the recombinant manufacture of papillomavirus L1 and/or L2 proteins and virus-like particles as well as their potential use as vaccines (WO 93/02189) have been described. Still further, recombinant papillomavirus capsid proteins purportedly capable of self-assembly into capsomere structures and viral capsids that comprise conformational antigenic epitopes have been reported (U.S. Pat. No. 5,437,951).

With respect to HPV capsid protein containing vaccines, it is widely accepted by those skilled in the art that a necessary prerequisite of an efficacious HPV L1 major capsid protein-based vaccine is that the L1 protein present conformational epitopes expressed by native human papillomavirus major capsid proteins (See, e.g., Hines et al, Gynecologic Oncology, 53:13-20 (1994); Suzich et al, Proc. Natl. Acad. Sci., U.S.A., 92:11553-11557 (1995)).

Both non-particle and particle recombinant HPV L1 proteins that present native conformational HPV L1 epitopes have been reported in the literature. It is known that L1 is stable in several oligomeric configurations, e.g., (i) capsomeres which comprise pentamers of the L1 protein and (ii) capsids which are constituted of seventy-two capsomeres in a T=7 icosahedron structure. Also, it is known that the L1 protein, when expressed in eukaryotic cells by itself, or in combination with L2, is capable of efficient self-assembly into capsid-like structures generally referred to as virus-like particles (VLPs).

VLPs have been reported to be morphologically and antigenically equivalent to authentic virions. Moreover, immunization with VLPs has been reported to elicit the production of virus-neutralizing antibodies. More specifically, results with a variety of animal papillomaviruses (canine oral papillomavirus and bovine papillomavirus-4) have suggested that immunization with VLPs results in protection against subsequent papillomavirus infection. Consequently, VLPs composed of HPV L1 proteins have been proposed as vaccines for preventing diseases associated with human papillomavirus infections.

The polyomaviruses are also non-enveloped double-stranded DNA viruses that were grouped, until recently, in the same family as papillomaviruses (e.g., family Papovaviridiae) because of their morphologic similarity. The polyomavirus capsid is also comprised of 72 pentamers of a single capsid protein (VP1) arranged on a T=7 icosahedral lattice, just as with the papillomavirus capsid. However, although VP1 proteins from various polyomaviruses have sequence similarity (L1 proteins from various papillomaviruses also have sequence similarity) there is no sequence similarity between VP1 and L1 proteins even though they yield a similar final structure. The mouse polyomavirus VP1 protein was the first of these proteins to be expressed and purified from E. coli, and shown to self assemble into VLPs. Thus, murine VP1 has served as the model for subsequent expression and structural studies with L1 and other VP1 proteins.

Polyomaviruses have been found in many animal species (e.g., SV40 in monkeys) but at present there are only four known human polyomaviruses: BKV, JCV, WU PyV, and KI PyV (the latter two were identified within the past year and their names are not yet finalized). A fifth virus, the lymphotropic polyomavirus (LPV) was first isolated from monkey cells but, recently, antibodies related to the LPV VP1 protein have been found in 15-20% of adult humans, suggesting that it or a relative is also a human virus. BKV and JCV are ubiquitous in humans, with 70-100% of populations tested being seropositive. These viruses appear to cause disease only when the person is immunosuppressed (JCV causes progressive multifocal leukoencephalopathy (PML); BKV causes hemorrhagic cystitis). For example, 5-10% of encephalopathies in AIDS patients are related to JCV. There are no current disease correlations for the other human viruses. Because of these known and potential disease associations, it would be desirable to develop both specific immunologic assays and potential vaccines for these viruses. VLPs would be the ideal candidates for both applications. The polyomavirus VP1 capsid is also very amenable to epitope insertions and modifications, and thus vaccine reagents to other viruses may be developed using the VP1 “backbone”. Finally, VLPs can be used to encapsidate both drugs and nucleic acids, making them interesting vehicles for gene therapy or drug delivery. Thus efficient production of VP1 would be desirable.

The production of recombinant proteins, such as viral capsid proteins, often results in low protein yields and/or protein insolubility. The difficulty in producing such proteins in Escherichia coli may be due to a number of reasons. First, the genes encoding these proteins show a high usage of synonymous codons considered rare in E. coli, and unavailability of the tRNAs corresponding to these rare codons may significantly limit the efficiency at which the genes are expressed {Makrides, 1996 #219}. Second, intracellular degradation pathways may exist for the recombinant proteins after expression in E. coli, possibly due to the intrinsic instability of the proteins in the host cell environment or the natural proteolytic system of E. coli {Leavitt, 1985 #130}. Finally, some major viral structural proteins form inclusion bodies when expressed in E. coli, resulting in low yields of soluble active proteins {Leavitt, 1985 #130; Chen, 2001 #134; Wrobel, 2000 #175}. Insoluble aggregates of proteins (inclusion bodies) require laborious isolation and purification procedures that increase costs, employ harsh reagents such as 8M urea, and further reduce product yields.

Therefore, there is a need in the art for improved methods for microbial expression of foreign proteins or polypeptides that can produce high yields and wherein the product can be readily purified from microbial lysates without altering the antigenicity or destroying the functional activity of the polypeptide. In particular, there exists a need in the art for improved production of viral major capsid proteins containing compositions that present conformational epitopes associated with native viruses.

### SUMMARY OF INVENTION

The present invention generally relates to improved processes for the production of recombinant proteins. The methods of the present invention are particularly useful for the production of proteins that are difficult to produce in bacterial expression systems. These aspects of the invention are based on the inventors' discovery of optimal time, temperature, media, culture and cell lysis conditions that result in increased protein yields and greatly simplify the purification of the protein. While the principles of the invention are generally applicable to recombinant protein expression, the invention is exemplified below via the production of a HPV L1-GST fusion protein and a MPV VP1 protein in E. coli.

One embodiment of the invention is a method for producing a protein including culturing bacteria until the bacteria reach stationary phase, wherein the bacteria express a recombinant nucleic acid molecule encoding the protein and wherein expression of the protein is inducible. The bacterial culture is cooled to about 25° C. and protein expression is induced by adding an induction agent. The bacteria are then cultured at about 25° C.

In one aspect of the invention, this embodiment includes the step of recovering the protein from the bacteria or the culture. This recovery may include purifying the protein, such as, by purifying the protein by chromatography, or on a GST column.

In one aspect, the protein recovered is a fusion protein, and in a preferred aspect, the fusion protein is a GST fusion protein. In a related aspect, the fusion protein is a viral protein fused to GST, and in a preferred aspect, the viral protein is a HPV or polyoma capsid protein, that may be a polyoma VP1 protein. In another preferred aspect, the fusion protein is GST-HPV L1 or GST-HPV L2.

In one embodiment, the bacteria are transfected with the recombinant nucleic acid molecule less than about 48 hours prior to culturing the bacteria until the bacteria reach stationary phase. In one aspect, culturing the bacteria until the bacteria reach stationary phase includes growing a starter culture of the bacteria.

In preferred embodiments, the bacteria may be E. coli, the bacteria are cultured in Terrific Broth, the recombinant nucleic acid molecule is a pGEX expression vector, the induction agent may be IPTG.

In one embodiment, the bacteria are cultured after the addition of an induction agent for at least about 4 hours.

Another embodiment is a method for producing a GST-HPV L1 fusion protein or a MPV VP1 protein including culturing E. coli until the E. coli reach stationary phase, wherein the E. coli express a recombinant nucleic acid molecule encoding the GST-HPV L1 fusion protein or the MPV VP1 protein and wherein expression of the protein is inducible by IPTG. The culture is cooled to about 25° C. and the protein expression is induced by adding IPTG. The bacteria are then cultured at about 25° C.

In one aspect, the method may also include recovering the protein from the E. coli or the culture. The recovering step may include purifying the GST-HPV L1 fusion protein, for example, by chromatography or on a GST column.

In one aspect, the E. coli are transfected with the recombinant nucleic acid molecule less than about 48 hours prior to culturing of the E. coli until the E. coli reach stationary phase. In another aspect, the culturing of the E. coli until the E. coli reach stationary phase includes growing a starter culture of the E. coli.

In one aspect, the recombinant nucleic acid molecule comprises a pGEX expression vector.

In another aspect, the bacteria are cultured for at least about 4 hours after the addition of the IPTG.

### BRIEF DESCRIPTION OF DRAWINGS

FIG. 1 shows the expression of soluble VP1 and GST-VP1 in Escherichia coli under optimal conditions: (L) Ladder; (a) untransformed RosDS cells-host proteins of 43 and 68 kDa are marked with asterisks; (b) induced RosDS-ptacVP1 (run 16, induction OD₆₀₀=0.5, 1.00 mM IPTG, 26° C. pre-induction temperature), and (c) induced RosDS-pGEXVP1 (run 12, induction OD₆₀₀=4.0, 1.00 mM IPTG, 26° C. pre-induction temperature).

FIG. 2 shows a half-normal probability plot. Effects (including interactions) selected for the model are labelled and represented as open squares.

FIG. 3 shows a response plot based on model of VP1 expression in Escherichia coli showing the effects of host strain (A), plasmid (B), induction OD₆₀₀ (C) and pre-induction temperature (E). IPTG concentration (D) was 0.05 mM in (A) and 1.00 mM in (B).

FIG. 4 shows the alignment of wild-type (M34958, pGEXVP1) and codon-optimized VP1 (pGEXVP1COpt) gene sequences. Rare codons (underlined) were identified with Codon Usage Analyzer 2.0 developed by Morris Maduro (lifesci.ucsb.edu/˜maduro/codonusage/usage2.0c.htm) using the database of Henaut and Danchin (Henaut and Danchin, 1996). Rare codons with tRNAs encoded by pLysSRARE in

RosDS cells are double-underlined.

FIG. 5 shows the expression of soluble VP1 from codon-optimized vector pGEXVP1COpt: (L) ladder; (a) soluble lysate of RosDS-pGEXVP1; (b) purified GST-VP1 expressed from pGEXVP1; (c) soluble lysate of RosDS-pGEXVP1COpt; (d) purified GST-VP1 expressed from pGEXVP1Copt. Cells were cultivated under the optimal conditions previously determined for RosDS-pGEXVP1 (run 12, induction OD₆₀₀=4.0, 1.0 mM IPTG, 26° C. pre-induction temperature).

FIG. 6 shows the transmission electron micrograph of virus-like particles assembled from VP1 protein expressed in Escherichia coli. Scale bar is 200 nm.

### DESCRIPTION OF EMBODIMENTS

Microbial protein expression systems typically involve the induction of protein expression at physiological temperatures (e.g., about 37° C. for bacterial systems) while the microorganisms are growing at an exponential rate (i.e., log-phase growth). Without being bound to any particular theory, it was believed that microorganisms in log-phase growth at physiological temperatures exhibit maximal rates of protein synthesis, including the increased synthesis of recombinant proteins. The present inventors have surprisingly found, however, that the induction of protein expression at lower than physiological temperatures after the microorganisms have reached a stationary growth phase can increase protein yields.

In one embodiment, the protein of interest can be produced by
a) culturing bacteria until the bacteria reach stationary phase, wherein the bacteria express a recombinant nucleic acid molecule encoding a protein and wherein expression of the protein is inducible;
b) cooling the culture to about 25° C.;
c) inducing protein expression by adding an induction agent; and
d) culturing the bacteria at about 25° C.

The methods disclosed herein provide numerous benefits over prior protein expression techniques. For example, the methods allow large amounts of protein to be quickly produced. In addition, the proteins produced using the methods, in particular viral structural proteins, may be soluble after cell lysis, thereby obviating the need for laborious and harsh purification conditions such as those employing ATP-urea.

The methods of the present invention can be used to express any protein for which the DNA sequence is known in the art. The methods disclosed herein may be particularly useful for increasing yields of proteins that are difficult to produce in bacterial expression systems. In some embodiments, the protein may be a viral protein, particularly a viral structural protein such as capsid protein. In certain embodiments, the viral protein may be a protein from a papillomavirus such as a human papillomavirus (HPV) or polyomavirus. In some embodiments, the protein may be a polyomavirus VP1 protein. In other embodiments, the protein may be a papillomavirus L1 or L2 protein. In some embodiments the protein is a fusion protein.

Any known polyomaviruses and papillomaviruses are suitable for use with the invention. Examples of suitable polyomaviruses include human polyomaviruses (e.g., BKV, JCV, WU PyV, and KI PyV), simian polyomaviruses (e.g., SV-12 and SV-40), murine polyomaviruses and bovine polyomaviruses. Suitable papillomaviruses include human papillomaviruses, simian papillomaviruses, murine papillomaviruses and bovine papillomaviruses. In some embodiments, the virus is a human polyomavirus or a human papillomavirus.

For example, in the case of HPV L1, DNAs have been reported in the literature and are publicly available. (See, e.g., Baker, Sequence Analysis of Papillomavirus, Genomes, pp. 321-384; Long et al, U.S. Pat. No. 5,437,931, Cole et al, J. Mol Biol., 193:599-608 (1987); Danos et al, EMBO J., 1:231-236 (1982); Cole et al J. Virol., 38(3):991-995 (1986).) Also, it is well known that HPV L1 DNAs exhibit significant homology. Therefore, a desired HPV L1 DNA can easily be obtained, e.g., by the use of a previously reported HPV L1 DNA or a fragment thereof as a hybridization probe or as a primer during polymerization chain reaction (PCR) amplification. Indeed, numerous HPV L1 DNAs have been cloned and expressed. Amino acid sequences of human papillomavirus proteins, as well as nucleotide sequences which encode the proteins, can be obtained from databases such as Genbank, Swiss-prot or EMBL.

HPV L1 or L2 DNA may be derived from any strain of HPV, such as from an HPV that is involved in cancer or condyloma acuminata, e.g., HPV-16, HPV-18, HPV-31, HPV-33, HPV-35, HPV-39, HPV-45, HPV-51, HPV-52, and HPV-56 are involved in cancer, and HPV-6, HPV-11, HPV-30, HPV-42, HPV-43, HPV-44, HPV-54, HPV-55, and HPV-70, are involved in warts.

Another example is the VP1 protein from polyomaviruses. VP1 DNA may be derived from any strain of polyomavirus, including the human polyomaviruses BKV, JCV, WU PyV, and KI PyV, or from murine polyomavirus (MPV). VP1 DNAs have been reported in the literature and are publicly available. (See, e.g., Eash et al, Cell. Mol. Life Sci. 63:865-876 (2006). VP1 DNA may also be obtained, e.g., by the use of a previously reported VP1 DNA or a fragment thereof as a hybridization probe or as a primer during polymerization chain reaction (PCR) amplification. Amino acid sequences of polyomavirus VP1 proteins, as well as nucleotide sequences which encode the proteins, can be obtained from databases such as Genbank, Swiss-prot or EMBL.

DNA segments encoding proteins can be inserted into expression vectors using standard molecular biology techniques. Briefly, a nucleic acid molecule encoding at least one desired protein is inserted into an expression vector in such a manner that the nucleic acid molecule is operatively linked to a transcription control sequence in order to be capable of effecting either constitutive or regulated expression of the nucleic acid molecule when transformed into a host cell. Nucleic acid molecules encoding one or more proteins can be on one or more expression vectors operatively linked to one or more transcription control sequences.

Many expression vectors are known in the art and are suitable for use in the present invention. According to the present invention, a recombinant vector is an engineered (i.e., artificially produced) nucleic acid molecule that is used as a tool for manipulating a nucleic acid sequence of choice and for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is nucleic acid sequences that are not naturally found adjacent to nucleic acid sequence to be delivered, although the vector can also contain regulatory nucleic acid sequences (e.g., promoters, untranslated regions) which are naturally found adjacent to nucleic acid molecules that are to be expressed or transferred by the host cells. The vector can be either RNA or DNA, either prokaryotic or eukaryotic, and typically is a plasmid. The vector can be maintained as an extrachromosomal element (e.g., a plasmid) or it can be integrated into the chromosome of the recombinant microorganism. The entire vector can remain in place within a host cell, or under certain conditions, the plasmid DNA can be deleted, leaving behind the nucleic acid molecule of the present invention. The integrated nucleic acid molecule can be under chromosomal promoter control, under native or plasmid promoter control, or under a combination of several promoter controls. Single or multiple copies of the nucleic acid molecule can be integrated into the chromosome. In one embodiment, a recombinant vector of the present invention contains at least one selectable marker for microorganisms according to the present invention. As used herein, the phrase “recombinant nucleic acid molecule” is used primarily to refer to a recombinant vector into which has been ligated the nucleic acid sequence to be cloned, manipulated, transformed into the host cell (i.e., the insert).

Typically, a recombinant vector, and therefore a recombinant nucleic acid molecule, includes at least one nucleic acid molecule operatively linked to one or more expression control sequences. As used herein, the phrase “recombinant molecule” or “recombinant nucleic acid molecule” primarily refers to a nucleic acid molecule or nucleic acid sequence operatively linked to a transcription control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to an expression control sequence in a manner such that the molecule can be expressed when transfected (i.e., transformed, transduced, transfected, conjugated or conduced) into a host cell. Expression control sequences include transcription control sequences, which are sequences that control the initiation, elongation, or termination of transcription. Particularly important transcription control sequences are those which control transcription initiation, such as promoter, enhancer, operator and repressor sequences. Suitable transcription control sequences include any transcription control sequence that can function in the bacterial cell being transformed. Recombinant nucleic acid molecules can also contain additional regulatory sequences, such as translation regulatory/control sequences, origins of replication, and other regulatory sequences that are compatible with the recombinant cell.

In one embodiment, a recombinant vector of the present invention is an expression vector. As used herein, the phrase “expression vector” is used to refer to a vector that is suitable for production of an encoded product (e.g., a protein of interest). In this embodiment, a nucleic acid sequence encoding the product to be produced is inserted into the recombinant vector to produce a recombinant nucleic acid molecule. The nucleic acid sequence encoding the protein to be produced is inserted into the vector in a manner that operatively links the nucleic acid sequence to regulatory sequences in the vector (e.g., a bacterial promoter) that enables the transcription and translation of the nucleic acid sequence within the recombinant microorganism.

It will be appreciated by one skilled in the art that use of recombinant DNA technologies can improve control of expression of transformed nucleic acid molecules by manipulating, for example, the number of copies of the nucleic acid molecules within the host cell, the efficiency with which those nucleic acid molecules are transcribed, the efficiency with which the resultant transcripts are translated, and the efficiency of post-translational modifications. Additionally, the promoter sequence might be genetically engineered to improve the level of expression as compared to the native promoter. Recombinant techniques useful for controlling the expression of nucleic acid molecules include, but are not limited to, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules to correspond to the codon usage of the host cell, and deletion of sequences that destabilize transcripts.

Recombinant nucleic acid molecules of the present invention, which can be either DNA or RNA, can also contain additional regulatory sequences, such as translation regulatory sequences, origins of replication, and other regulatory sequences that are compatible with the recombinant cell. In one embodiment, a recombinant molecule of the present invention, including those which are integrated into the host cell chromosome, also contains secretory signals (i.e., signal segment nucleic acid sequences) to enable an expressed protein to be secreted from the cell that produces the protein. Suitable signal segments include a signal segment that is naturally associated with the protein to be expressed or any heterologous signal segment capable of directing the secretion of the protein according to the present invention. In another embodiment, a recombinant molecule of the present invention comprises a leader sequence to enable an expressed protein to be delivered to and inserted into the membrane of a host cell. Suitable leader sequences include a leader sequence that is naturally associated with the protein, or any heterologous leader sequence capable of directing the delivery and insertion of the protein to the membrane of a cell.

One example of an expression system suitable for use in the present invention is a GST fusion vector. The pGEX system, for example, utilizes glutathione S-transferase as an N-terminal fusion partner (Smith et al., Gene 67:31-40 (1988)). The system provides an easy way to purify recombinant protein using glutathione immobilized on a chromatography gel. Fusion proteins containing GST-tags at the N-terminus of the protein are also described in U.S. Pat. No. 5,654,176 (additional information may be obtained from GE Healthcare, USA). Many other expression vectors and systems are known in the art and are suitable for use with the present invention. Examples include the pET expression system (Novagen, USA), His-tagged expression systems and intein fusion expression systems (New England Biolabs, USA).

Recombinant vectors may be introduced into a microbial host cell by standard techniques, including, but not limited to, transformation, transfection, particle bombardment, electroporation, microinjection, lipofection, adsorption, or infection. According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells, such as algae, bacteria and yeast, or into plant cells. In microbial systems and plant systems, the term “transformation” is used to describe an inherited change due to the acquisition of exogenous nucleic acids by the microorganism or plant and is essentially synonymous with the term “transfection.” Therefore, transfection techniques include, but are not limited to, transformation, chemical treatment of cells, particle bombardment, electroporation, microinjection, lipofection, adsorption, infection and protoplast fusion.

A recombinant cell may be produced by transforming a bacterial or yeast cell (i.e., a host cell) with one or more recombinant molecules, each comprising one or more nucleic acid molecules operatively linked to an expression vector containing one or more transcription control sequences. The phrase, operatively linked, refers to insertion of a nucleic acid molecule into an expression vector in a manner such that the molecule is able to be expressed when transformed into a host cell. As used herein, an expression vector is a DNA or RNA vector that is capable of transforming a host cell and of effecting expression of a specified nucleic acid molecule. Preferably, the expression vector is also capable of replicating within the host cell. In the present invention, expression vectors are typically plasmids. Expression vectors of the present invention include any vectors that function (i.e., direct gene expression) in a yeast host cell or a bacterial host cell, preferably an Escherichia coli host cell.

A microorganism to be used in methods of the present invention (e.g., a host cell or production organism) is any microorganism (e.g., a bacterium, a protist, an alga, a fungus, or other microbe), and is most preferably a bacterium, a yeast or a fungus. Suitable bacterial genera include, but are not limited to, Escherichia, Bacillus, Lactobacillus, Pseudomonas and Streptomyces. Suitable bacterial species include, but are not limited to, Escherichia coli, Bacillus subtilis, Bacillus licheniformis, Lactobacillus brevis, Pseudomonas aeruginosa and Streptomyces lividans. Suitable genera of yeast include, but are not limited to, Saccharomyces, Schizosaccharomyces, Candida, Hansenula, Pichia, Kluyveromyces, and Phaffia. Suitable yeast species include, but are not limited to, Saccharomyces cerevisiae, Schizosaccharomyces pombe, Candida albicans, Hansenula polymorpha, Pichia pastoris, P. canadensis, Kluyveromyces marxianus and Phaffia rhodozyma. Suitable fungal genera include, but are not limited to, Aspergillus, Absidia, Rhizopus, Chrysosporium, Neurospora and Trichoderma. Suitable fungal species include, but are not limited to, Aspergillus niger, A. nidulans, Absidia coerulea, Rhizopus oryzae, Chrysosporium lucknowense, Neurospora crassa, N. intermedia and Trichoderm reesei. Although Escherichia coli is one preferred bacteria and is used to exemplify various embodiments of the invention, it is to be understood that any microorganism capable of expressing foreign proteins can be utilized. It is to be appreciated that a number of the species described above include a variety of subspecies, types, subtypes, etc. that are meant to be included within the aforementioned species.

Suitable microorganisms may be cultured using standard protocols, media and aeration. Examples of media that may be used to culture Escherichia coli include 2X YT, Luria Broth (LB) and Terrific Broth (TRB). Typically, a microorganism is transformed with the expression vector of interest and plated to isolate single colonies each time the method is carried out. For example, freshly transformed competent bacterial cells can be incubated on antibiotic containing plates overnight. Antibiotic resistance and other means of microbial selection are well known in the art. Isolated colonies that grow on the selection media may then be used to inoculate cultures for protein production.

In some embodiments, starter cultures are produced for subsequent inoculation of larger cultures. For example, an isolated bacterial colony may be used to inoculate a relatively small amount of media, which in turn is used to inoculate a larger main culture. In certain embodiments, the starter culture is cultured at a temperature from about 30° C. to about 37° C. In some embodiments, the starter culture is cultured at a temperature of about 30° C.; in other embodiments, at a temperature of about 37° C.

Microorganisms containing the expression vector of interest are cultured at physiological temperatures and the growth (e.g., using optical density (OD) measurements at 595 nm) is monitored periodically until the cells reach stationary phase growth. For example, a two-liter shake flask culture of bacteria cultured in TRB will reach stationary phase at an OD₅₉₅ of about 4.0. As used herein, “stationary phase” and “stationary phase growth” refer to a microorganism growth rate wherein the doubling time of the microorganism is not logarithmic (i.e., not log-phase growth). For example bacteria typically exhibit a doubling time of about 20-30 minutes in log-phase growth, where as the doubling time is about 2-4 hours in stationary phase growth. One of skill in the art will appreciate that the growth phase of microorganisms may be measured by various means and that measured values will vary with parameters such as the microorganism, culture conditions, and the like.

Once the microorganisms reach stationary phase, the culture may be cooled to a temperature of from about 22° C. to about 25° C. In some embodiments, the culture may be cooled to a temperature of about 22° C.; in other embodiments, to a temperature of about 22° C. Any cooling apparatus suitable for use with microorganism cultures may be used.

Following cooling, the expression of the protein of interest by the microorganisms is induced by the addition of the appropriate stimulus. The stimulus provided will vary depending on the expression vector used to express the protein. One example is the addition of a chemical (for example, IPTG) to the culture media. For example, E. coli expression of a HPV L1-GST fusion protein using a pGEX vector is induced by the addition of IPTG to the culture as described in published protocols. After induction, the microorganisms are cultured for a period of time at the same temperature to allow for protein production. The amount of time will vary depending upon factors such as the microorganism cultured, the protein expressed and the like. One of skill in the art can readily determine the optimal production time by monitoring the density of the culture and/or removing samples from the culture and examining the amount of the desired protein present using assays standard in the art. By way of example, E. coli expressing HPV L1-GST fusion proteins are typically cultured in a shake flask at about 25° C. for about six hours, until the OD₅₉₅ reaches about 8.0.

Microorganisms of the present invention can be cultured in conventional shake flasks or in aerated bioreactors. The microorganisms can be cultured by any fermentation process which includes, but is not limited to, batch, fed-batch, cell recycle, and continuous fermentation. Although the present invention is exemplified on a laboratory scale, one of skill in the art will appreciate that the methods described herein may be employed in large scale or industrial microbial fermentation systems. One of skill in the art will also appreciate that values such as optical density (OD) will vary depending upon the particular culture or fermentation system used. For example, higher OD values may be achieved in large-scale fermentation vessels in comparison to laboratory-scale shake flask cultures.

Sufficient oxygen must be transferred to the medium during the course of the fermentation to maintain cell growth during the initial cell growth and to maintain metabolism, and protein production. Oxygen is conveniently provided by agitation and aeration of the medium. Conventional methods, such as stirring or shaking, may be used to agitate and aerate the medium. Preferably the oxygen concentration in the medium is greater than about 15% of the saturation value (i.e., the solubility of oxygen in the medium at atmospheric pressure and about 30-40° C.) and more preferably greater than about 20% of the saturation value, although excursions to lower concentrations may occur if fermentation is not adversely affected. It is further understood that the oxygen level can be allowed to reach very low levels for any appropriate amount of time during the fermentation if it enhances stability and formation of protein during the production process. The oxygen concentration of the medium can be monitored by conventional methods, such as with an oxygen electrode. Other sources of oxygen, such as undiluted oxygen gas and oxygen gas diluted with inert gas other than nitrogen, can be used.

In some instances, the protein may be recovered, and in others, the cell may be harvested in whole (e.g., for ex vivo administration), either of which can be used in a composition. A preferred cell to culture is any suitable host cell as described above. Effective culture conditions include, but are not limited to, effective media, bioreactor, temperature, pH and oxygen conditions that permit protein production and/or recombination. An effective medium refers to any medium in which a given host cell is typically cultured. Such medium typically comprises an aqueous medium having assimilable carbon, nitrogen and phosphate sources, and appropriate salts, minerals, metals and other nutrients, such as vitamins. Cells can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

In certain embodiments, the methods of the present invention further include a step of recovering the protein from the cultured cells. Depending on the vector and host system used for production, resultant proteins of the present invention may either remain within the recombinant cell; be secreted into the culture medium; be secreted into a space between two cellular membranes; or be retained on the outer surface of a cell membrane. The phrase “recovering the protein” refers to collecting the whole culture medium containing the protein and need not imply additional steps of separation or purification.

In certain embodiments, the protein may be purified from the culture medium. In additional embodiments, the cultured cells are separated from the culture medium using standard techniques (e.g., by centrifugation). In some embodiments, the cells are stored (e.g., frozen) for future use. A lysate may be prepared from the cells using conventional methods. In some embodiments, the cells are lysed by resuspension in a lysis buffer; in others, the cells are lysed mechanically (e.g., using a high-pressure homogenizer), or by a combination of these methods. The homogenate may be separated from insoluble cell components by techniques such as centrifugation or filtration to produce a cleared lysate. The techniques described above are well known in the art.

Proteins produced according to the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization. In certain embodiments, the protein is purified on a GST column. Methods for purifying proteins on GST affinity columns are well known to those of skill in the art. In embodiments where the protein is expressed as a fusion protein, protein of interest (e.g., HPV L1) can be separated from the fusion partner (e.g., GST) by conventional techniques that may vary based on the expression system used. For example, the fusion partner may be separated by thrombin cleavage or factor Xa cleavage.

In one aspect, the present invention relates to the expression of a viral capsid protein, preferably that of a non-enveloped virus. In some embodiments, the viral capsid protein is a papillomavirus L1 or L2 capsid protein or a polyomavirus VP1 capsid protein, or fragment thereof, expressed as a glutathione-S-transferase (GST) fusion protein. The GST protein may be fused at the amino-terminal or carboxy-terminal portion of the viral capsid protein or fragment thereof In some embodiments, the GST protein is fused to the amino terminus of an intact or carboxy-truncated HPV L1 protein.

The proteins produced by the methods of the present invention may be used for any method or application for which the protein may be employed. In the case of viral capsid proteins such as VP1 or viral capsid fusion proteins such as HPV L1-GST, the proteins may be used to form pentamers (capsomeres) or VLPs. A detailed discussion of viral capsid fusion proteins and the production of pentamers (capsomeres) or VLPs can be found in International Publication No. WO 02/04007, the contents of which are hereby incorporated by reference in their entirety.

The proteins of the present invention, such as HPV L1 or polyomavirus VP1, and fusions, capsomeres and VLPs thereof, can be used in vaccine preparations or as therapeutic compositions. The method of use of the therapeutic composition or vaccine of the present invention preferably elicits an immune response in an animal such that the animal is protected from the disease or condition (including infection), or from symptoms resulting from the disease or condition (including infection). As used herein, the phrase “protected from a disease” refers to reducing the symptoms of the disease; reducing the occurrence or reoccurrence of the disease, and/or reducing the severity of the disease. Protecting an animal can refer to the ability of a composition of the present invention, when administered to an animal, to prevent a disease from occurring and/or to cure or to alleviate disease symptoms, signs or causes. As such, to protect an animal from a disease includes both preventing disease occurrence (prophylactic treatment or prophylactic vaccine) and treating an animal that has a disease or that is experiencing initial symptoms of a disease (therapeutic treatment or a therapeutic vaccine). In particular, protecting an animal from a disease is accomplished by eliciting an immune response in the animal by inducing a beneficial or protective immune response that may, in some instances, additionally suppress (e.g., reduce, inhibit or block) an overactive or harmful immune response. The term, “disease” refers to any deviation from the normal health of an animal and includes a state when disease symptoms are present, as well as conditions in which a deviation (e.g., infection) has occurred, but symptoms are not yet manifested.

In one embodiment, any of the vaccines of the present invention is administered to an individual, or to a population of individuals, who have been infected with a pathogen, such as a papillomavirus or a polyomavirus. In another embodiment, any of the vaccines of the present invention is administered to an individual, or to a population of individuals, who are at risk of being infected with such a pathogen. Such individuals can include populations identified as higher-risk for papillomavirus or polyomavirus infection than, for example, the normal or entire population of individuals. Such individuals can also include populations that are selected for a particular vaccine of the present invention due to expected pathogen strains (e.g., viral strains) in the geographical location of the population. Such populations can be defined by any suitable parameter. In another embodiment, any of the vaccines of the present invention is administered to any individual, or to any population of individuals, regardless of their known or predicted infection status or susceptibility to becoming infected with a particular pathogen.

In some embodiments, vaccines of the present invention may comprise papillomavirus L1 or L2 proteins, polyomavirus VP1 proteins, or combinations thereof. In certain embodiments, the vaccines may comprise VLPs comprising L1, L2, or VP1 proteins or combinations thereof.

In addition to vaccines for papillomavirus and polyomavirus, VLPs of the present invention may be used to deliver immunogenic epitopes from other pathogens. For example, foreign protein epitopes may be appended to VLPs (e.g., by using standard recombinant DNA techniques to express a fusion between a viral capsid protein and a peptide or protein from a pathogen of interest), and the VLPs may be used as a vaccine for the pathogen from which the epitope was derived. Epitopes from any pathogen may be used, including viral capsid proteins or other epitopes from influenza virus, HIV, RSV or the like. Adjuvant molecules may also be linked to the VLPs to increase immunogenicity.

VLPs may also be used as delivery vehicles for DNA or drugs. Drug molecules or nucleic acids may be encapsidated within VLPs, for example, by using in vitro VLP assembly reactions in the presence of the drug or nucleic acid molecules. VLPs may also be modified to contain heterologous peptide sequences that can retarget the VLPs to particular cells or tissues. For example, immunoglobulin-binding domains may be incorporated into VP1 VLPs. Antibodies specific for a particular cell population may then be coupled to the VLPs to target the VLPs (as well as any drug or nucleic acid molecule encapsidated therein) to the cell population (Gleiter et al., Biol. Chem. 384:247-255 (2003)). A review of the use of VLPs as delivery vessels and as vaccines papillomaviruses, polyomaviruses, and other pathogens can be found in Garcea et al., Curr. Opin. Biotech. 15:513-517 (2004), the contents of which are hereby incorporated by reference in their entirety.

More specifically, a vaccine as described herein, when administered to an animal by the method of the present invention, preferably produces a result which can include alleviation of the pathogen infection (e.g., reduction of at least one symptom or clinical manifestation of the infection), elimination of the infection or reduction in the time to eliminate the infection, prevention of the infection and/or symptoms related thereto, and stimulation of effector cell immunity against the infection, as well as humoral immunity. In addition, the vaccine preferably primes the immune system to prevent or reduce all infection by the pathogen, including all life cycle forms, strains, or mutants of the pathogen, whether free in the circulation or in the cells or tissues of an individual. The vaccine also preferably confers long-lasting immunity against the pathogen, or at least a universal or cross-protective immunity, so that future infections by new strains or mutants are more readily prevented and/or eliminated.

The present invention includes the delivery of a composition or vaccine of the invention to an animal. The administration process can be performed ex vivo or in vivo. Ex vivo administration refers to performing part of the regulatory step outside of the patient, such as administering a composition of the present invention to a population of cells (dendritic cells) removed from a patient under conditions such that a delivery vehicle and antigen are loaded into the cell, and returning the cells to the patient. The therapeutic composition of the present invention can be returned to a patient, or administered to a patient, by any suitable mode of administration.

Administration of a vaccine or composition, alone or in combination with a carrier according to the present invention, can be conducted by any route. The preferred routes of administration will be apparent to those of skill in the art. Preferred methods of administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracranial, intraspinal, intraocular, aural, intranasal, oral, pulmonary administration, impregnation of a catheter, and direct injection into a tissue.

According to the present invention, an effective administration protocol (i.e., administering a vaccine or therapeutic composition in an effective manner) comprises suitable dose parameters and modes of administration that result in elicitation of an immune response in an animal that has a disease or condition, or that is at risk of contracting a disease or condition, preferably so that the animal is protected from the disease. Effective dose parameters can be determined using methods standard in the art for a particular disease. Such methods include, for example, determination of survival rates, side effects (i.e., toxicity) and progression or regression of disease. In accordance with the present invention, a suitable single dose size is a dose that is capable of eliciting an antigen-specific immune response in an animal when administered one or more times over a suitable time period. Doses can vary depending upon the disease or condition being treated.

One aspect of the present invention involves the development of HPV type-specific vaccines. The vaccines of the invention will contain an amount of the subject stable HPV capsomeres sufficient to induce formation of neutralizing antibodies in the host contained in a pharmaceutically acceptable carrier.

The vaccines will be administered in therapeutically effective amounts. That is, in amounts sufficient to produce a protective immunological response. Generally, the vaccines will be administered in dosages ranging from about 0.1 mg protein to about 20 mg protein, more generally about 0.001 mg to about 100 mg protein. A single or multiple dosages can be administered.

The method of the present invention makes possible the preparation of HPV capsomere containing vaccines for preventing papillomavirus infection. Further, by following the methods of the invention, vaccines for any immunogenic type of human specific papillomavirus can be made.

As more than one HPV type may be associated with HPV infections, the vaccines may comprise stable HPV capsomeres derived from more than one type of PV. For example, as HPV 16 and 18 are associated with cervical carcinomas, therefore a vaccine for cervical neoplasia may comprise capsomere of HPV 16; of HPV 18; or both HPV 16 and 18.

In fact, a variety of neoplasia are known to be associated with PV infections. For example, HPVs 3a and 10 have been associated with flat warts. A number of HPV types have been reported to be associated with epidermodysplasia verruciformis (EV) including HPVs 3a, 5, 8, 9, 10, and 12. HPVs 1, 2, 4, and 7 have been reported to be associated with cutaneous warts and HPVs 6b, 11a, 13, and 16 are associated with lesions of the mucus membranes. See, for example, Kremsdorf et al, J. Virol., 52:1013-1018 (1984); Beaudenon et al, Nature, 321:246-249 (1986); Heilman et al, J. Virol., 36:395-407 (1980); and DeVilliers et al, J. Virol., 40:932-935 (1981). Thus, the subject vaccine formulations may comprise a mixture of capsomere from different HPV types depending upon the desired protection.

HPV capsomeres can also be utilized for serotyping and for incorporation in serotyping kits. For serological testing, the kits will comprise the subject HPV capsomere and means for detection such as enzyme substrates, labeled antibody, and the like.

In another embodiment, HPV capsomeres of the present invention can be utilized to detect, diagnose, serotype, and treat papillomavirus infection. When used for diagnosis or serotyping, capsomeres according to the invention may be labeled using any of a variety of labels and methods of labeling. Examples of types of labels which can be used in the present invention include, but are not limited to, enzyme labels, radioisotopic labels, non-radioactive isotopic labels, fluorescent labels, toxin labels, and chemiluminescent labels.

Another embodiment of the present invention relates to compositions comprising any of the proteins produced by the methods disclosed herein or a nucleic acid encoding the protein. Such a composition of the present invention can include any carrier with which the protein or nucleic acid molecule is associated by virtue of the protein or nucleic acid molecule preparation method, a purification method, or a preparation of the protein or nucleic acid molecule for use in an in vitro, ex vivo, or in vivo method according to the present invention. For example, such a carrier can include any suitable excipient, buffer and/or delivery vehicle, such as a pharmaceutically acceptable carrier (discussed below), which is suitable for combining with the protein or nucleic acid molecule of the present invention so that the protein or nucleic acid molecule can be used in vitro, ex vivo or in vivo according to the present invention.

The composition typically also includes a pharmaceutically acceptable carrier. According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. Preferred pharmaceutically acceptable carriers are capable of maintaining a protein or recombinant nucleic acid molecule of the present invention in a form that, upon arrival of the protein or recombinant nucleic acid molecule at the cell target in a culture or in patient, the protein or recombinant nucleic acid molecule is capable of interacting with its target.

Suitable excipients of the present invention include excipients or formularies that transport or help transport, but do not specifically target a composition to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Compositions of the present invention can be sterilized by conventional methods and/or lyophilized.

One type of pharmaceutically acceptable carrier includes a controlled release formulation that is capable of slowly releasing a composition of the present invention into a patient or culture. As used herein, a controlled release formulation comprises a compound of the present invention (e.g., a protein (including homologues), an antibody, a nucleic acid molecule, or a mimetic) in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers of the present invention include liquids that, upon administration to a patient, form a solid or a gel in situ. Preferred carriers are also biodegradable (i.e., bioerodible). When the compound is a recombinant nucleic acid molecule, suitable delivery vehicles include, but are not limited to liposomes, viral vectors or other delivery vehicles, including ribozymes. Natural lipid-containing delivery vehicles include cells and cellular membranes. Artificial lipid-containing delivery vehicles include liposomes and micelles. A delivery vehicle of the present invention can be modified to target to a particular site in a patient, thereby targeting and making use of a compound of the present invention at that site. Suitable modifications include manipulating the chemical formula of the lipid portion of the delivery vehicle and/or introducing into the vehicle a targeting agent capable of specifically targeting a delivery vehicle to a preferred site, for example, a preferred cell type. Other suitable delivery vehicles include gold particles, poly-L-lysine/DNA-molecular conjugates, and artificial chromosomes.

A pharmaceutically acceptable carrier which is capable of targeting is herein referred to as a “delivery vehicle.” Delivery vehicles of the present invention are capable of delivering a composition of the present invention to a target site in a patient. A “target site” refers to a site in a patient to which one desires to deliver a composition. For example, a target site can be any cell which is targeted by direct injection or delivery using liposomes, viral vectors or other delivery vehicles, including ribozymes. Examples of delivery vehicles include, but are not limited to, artificial and natural lipid-containing delivery vehicles, viral vectors, and ribozymes. Natural lipid-containing delivery vehicles include cells and cellular membranes. Artificial lipid-containing delivery vehicles include liposomes and micelles. A delivery vehicle of the present invention can be modified to target to a particular site in a mammal, thereby targeting and making use of a compound of the present invention at that site. Suitable modifications include manipulating the chemical formula of the lipid portion of the delivery vehicle and/or introducing into the vehicle a compound capable of specifically targeting a delivery vehicle to a preferred site, for example, a preferred cell type. Specifically, targeting refers to causing a delivery vehicle to bind to a particular cell by the interaction of the compound in the vehicle to a molecule on the surface of the cell. Suitable targeting compounds include ligands capable of selectively (i.e., specifically) binding another molecule at a particular site. Examples of such ligands include antibodies, antigens, receptors and receptor ligands. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

One preferred delivery vehicle of the present invention is a liposome. A liposome is capable of remaining stable in an animal for a sufficient amount of time to deliver a nucleic acid molecule described in the present invention to a preferred site in the animal. A liposome, according to the present invention, comprises a lipid composition that is capable of delivering a nucleic acid molecule described in the present invention to a particular, or selected, site in a patient. A liposome according to the present invention comprises a lipid composition that is capable of fusing with the plasma membrane of the targeted cell to deliver a nucleic acid molecule into a cell. Suitable liposomes for use with the present invention include any liposome. Preferred liposomes of the present invention include those liposomes commonly used in, for example, gene delivery methods known to those of skill in the art. More preferred liposomes comprise liposomes having a polycationic lipid composition and/or liposomes having a cholesterol backbone conjugated to polyethylene glycol. Complexing a liposome with a nucleic acid molecule of the present invention can be achieved using methods standard in the art.

Another preferred delivery vehicle comprises a viral vector. A viral vector includes an isolated nucleic acid molecule useful in the present invention, in which the nucleic acid molecules are packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses and retroviruses.

The present invention also encompasses antibodies specific for proteins produced by the methods disclosed herein, as well as methods of producing the antibodies. The proteins of the present invention may be introduced into a suitable host animal using standard techniques to elicit an immune response in the host. Polyclonal or monoclonal antibodies may be isolated from the host using techniques known in the art.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies, humanized antibodies, antibodies that can bind to more than one epitope (e.g., bi-specific antibodies), or antibodies that can bind to one or more different antigens (e.g., bi- or multi-specific antibodies), may also be employed in the invention.

Various aspects of the present invention are described in the following experiments. These experimental results are for illustrative purposes only and are not intended to limit the scope of the present invention.

### Examples

The following Materials and Methods were used in the examples below.

### Bacterial Strains, Media, and Buffers

BL21 CodonPlus(DE3)-RIL Competent Cells were from Stratagene (Cat #230245).

For Ampicillin/Choramphenicol (AMP/CAM) plates, tryptone (10 g), yeast extract (5 g), NaCl (10 g) and agar (10 g) are added to 1 liter of MilliQ water, and the resulting medium is sterilized by autoclaving at 121° C. for 20 minutes. The solution is allowed to cool to 45° C., then 1 ml of ampicillin stock solution (50 mg/ml) and 1 ml of Chloramphenicol stock solution (34 mg/ml) is added. The medium is mixed by swirling and poured into plates, allowing about 25 ml of medium per plate.

Terrific Broth (TRB) was made according to Molecular Cloning Lab Manual, 3^{rd} Edition, pg A2.4. Tryptone (12 g), yeast extract (24 g) and 50% Glycerol (8 ml) is added to 900 ml of deionized H₂O, mixed until the solutes have dissolved and then sterilized by autoclaving for 20 minutes at 15 psi on liquid cycle (cycle #10). The solution is allowed to cool to 60° C. or less, and then 100 ml of a sterile solution of 0.17 M KH₂PO₄, 0.72 M K₂HPO₄ is added (this solution is made by dissolving 2.31 g of KH₂PO₄ and 12.54 g of K₂HPO₄ in 90 ml of deionized H₂O. After the salts have dissolved, the volume of the solution is adjusted to 100 ml with deionized H₂O and sterilize by autoclaving for 20 minutes at 15 psi on liquid cycle.)

Buffer L was made according to the following recipe:

| | | |
|---|---|---|
| Stock | Final conc | Amount added for 1 L |

| | | | | |
|---|---|---|---|---|
| 2M Tris pH 8.0 | 40 | mM | 20 | ml |
| 5M NaCl | 200 | mM | 40 | ml |
| 0.5M EDTA pH 8.0 | 1 | mM | 2 | ml |
| 50% Glycerol | 5% | | 100 | ml |

| | | | |
|---|---|---|---|
| DTT | 5 | mM | 5 ml (add just before use) |

After adding deionized H₂O to a final volume of 1 liter, buffer L is filtered using a 0.22 μm filter.

### Example 1

The following example demonstrates the production of a GST-fused HPV L1 protein using the methods described above.

BL21 CodonPlus(DE3)-RIL Competent Cells were transformed with the expression vector pGEX-4T-2 (available from GE Healthcare) into which the DNA encoding HPV 11 L1 protein was cloned (Finnen et al., J Virol. 77, 4818-4826 (2003)). The cells were spread onto AMP/CAM plates and incubated overnight at 37° C.

A started culture was produced by picking an isolated colony from a freshly streaked plate and culturing it in a tube containing 5 ml TRB plus AMP (50 μg/ml) and CAM (34 μg/ml) for 16 hours at 30° C. with agitation (180 rpm). After 16 hours, the OD₅₉₅ was read, and should be in the range of 2.5-3.0.

For the main cultures, 400 μl of starter culture was added to 2 L baffled flasks containing 400 ml each of TRB (360 ml of medium+40 ml of 0.17 M KH₂PO₄, 0.72 M K₂HPO₄), 400 μl AMP (50 mg/ml) and 400 μl CAM (34 mg/ml). The cultures were incubated at 37° C. and 180 rpm, and the OD₅₉₅ was monitored hourly. When the OD₅₉₅ of the cultures reached 4.0 (approx. 6 hrs), the cultures were cooled to 25° C. in an ice-water bath.

L1 protein expression was induced by adding 160 μl of 0.5M of IPTG (0.2 mM final concentration) to each flask. The cultures were then allowed to grow at 25° C. and 180 rpm until the OD₅₉₅ reached 8.0 (approx. 6 hrs).

The cell pellets were then harvested and weighed. 250 ml centrifuge bottles were weighed, filled with culture media, then centrifuged at 2400 g for 30 minutes at 4° C. The supernatant was poured off, the pellets drained, and the wet weight of each pellet determined. Cell pellets were stored at −70° C. before further processing.

Cell pellets (from 400 ml of culture) were resuspended in 200 ml of Buffer L and PMSF was added to a final concentration of 1 mM (2 ml of 100 mM/200 ml) along with 1 protease inhibitor cocktail tablet (Roche Diagnostics REF #11 873 580 001) and 1 ml of 0.5M DTT (5 mM final). The cell suspension was kept on ice and the cells passed through a French Press at 1000 psig twice to ensure complete cell lysis. The cell lysate was centrifuged at 22,000 g for 30 minutes at 4° C. and the clarified lysate collected and filtered using a 0.22 μm filters.

### Example 2

The following example demonstrates the column purification of a GST-fused HPV L1 protein.

The filtered supernatant produced in Example 1 can be applied immediately to a GST column or stored on ice at 4° C. for at least a week. Lysate that has been stored at 4° C. overnight or longer is passed over a 0.2 μm filter before loading onto the GST column.

A 5 ml GST-TRAP FF column (GE Healthcare Cat #17-5131-01) was used to capture the fusion protein at room temperature. The column was equilibrated with Buffer L (0.2 μm filtered and degassed), and a 50 ml Superloop (GE Healthcare Cat #19-7850-01) was used to load the column. 50 ml of the lysate was poured over the 5 ml column, collecting the flow thru. The column was eluted with Buffer L+10 mM reduced Glutathione. The column was then stripped with 6M Guanidine-HCl and re-equilibrated with Buffer L before and a second 50 ml aliquot of lysate was applied. The flow rate for equilibration, loading and elution was 0.5 ml/min, while the fraction size for 5 ml column was 1 ml. Approximately 2.5 mg of protein from each 50 ml of lysate was recovered. An additional 1 mg may be recovered by passing 50 ml of flow thru back over the column. In additional experiments, 3 mg of protein was recovered after running 80 ml of lysate over the 5 ml column before eluting. Running 100 ml of flow thru over the column before eluting allowed the recovery of 2 mg of protein from those fractions.

### Example 3

The following example demonstrates the production of a VP1 protein using the methods described above.

VP1 of murine polyomavirus was used as a model to study expression of soluble viral structural protein in E. coli. Polyomavirus is a non-enveloped, icosahedral DNA virus that is closely related to the human papillomavirus. A VLP of murine polyomavirus is a 50 nm icosahedral (T=7d) structure (Rayment et al., 1982) formed from 72 capsomeres, each of which is a pentamer of the 42.5 kDa protein VP1 (Salunke et al., 1986). Different strategies to express structural proteins of Papovavirus in E. coli have been reported in the literature. It has been shown that expression yield can be enhanced with tags or fusion partners such as His-tag (Braun et al., 1999), glutathione-S-transferase (GST) (Chen et al., 2001), or a modified Saccharomyces cerevisae intein (Schmidt et al., 2000). The utilization of fusion tags also improves the ease and efficiency of downstream purification procedures. In addition, cell cultures were induced at room temperature to improve solubility of expressed HPV L1 (Chen et al., 2001). E. coli strains deficient in the GroELS chaperone system have also been used to increase the production of soluble His-VP1 of SV40 (Wrobel et al., 2000).

Expression optimization in E. coli performed by varying the experimental variable one at a time is inefficient. Design of experiment (DOE) (Montgomery, 2005) provides a more reliable and effective methodology that allows interactions among several factors to be determined (Nikerel et al., 2005, Swalley et al., 2006, Wang et al., 2005), and is thus used in this work to study the effect of host, plasmid and culture growth conditions on the expression of viral structural protein in E. coli. The factorial experimental design used in this study identified the most significant factors affecting the production of VP1 in E. coli and successfully increased VP1 expression by an order of magnitude compared to reported yields. These results suggested that the production of VP1 in E. coli is affected by rare codon effect, which can be overcome by the use of an E. coli host strain that co-expresses several cognate tRNAs.

Plasmid Construction and Host Strains

The plasmids pGEXVP1 and ptacVP1 (pALVP₁TAC (Leavitt et al., 1985)) were used for VP1 protein expression. pGEXVP1 was constructed by inserting the murine polyomavirus VP1 sequence (M34958) between the BamHI and XhoI sites of a pGEX-4T-1 vector (GE Healthcare Biosciences, Chalfont St. Giles, UK), allowing the expression of GST-tagged VP1. Both vectors were generously donated by Professor Robert Garcea (University of Colorado, Colo., USA). VP1 gene with optimized codon usage was designed and synthesized by Geneart AG (Regensberg, Germany) with GeneOptimizer™ (Geneart AG) and inserted into pGEX-4T-1 as described above to create pGEXVP1COpt. Escherichia coli cells RB791 was purchased from American Type Culture Collection (VA, USA) and Rosetta(DE3)pLysS (RosDS) from Novagen (CA, USA). Preparation of chemically competent cells by calcium chloride-mediated method and cell transformation with heat shock treatment were performed as previously described (Sambrook and Russell, 2001).

Experimental Design

A 2⁵ full factorial design was used to study the effects of bacterial strain (A, RosDS or RB791), expression vector (B, pGEXVP1 or ptacVP1), cell density (measured as optical density at 600 nm) at induction (C, 0.5 or 4.0), isopropyl-β-D-thiogalactoside (IPTG) concentration (D, 0.05 or 1 mM) and pre-induction cultivation temperature (E, 37° C. or 26° C.) on the expression of soluble VP1 in E. coli. Experimental design and data analysis were performed with Design Expert (version 7.1, Stat-Ease MN, USA) using a methodology previously described (Montgomery, 2005). The total design matrix with 32 experiments in four blocks is presented in Table 1. Response variable in the optimization study was defined as the normalized expression level of soluble VP1 (Y_{S}) in mgL⁻¹OD⁻¹(milligrams per liter of culture at OD₆₀₀ of 1), which bears the same significance as the yield of product per unit biomass. The actual concentration of VP1 in the fusion form (GST-VP1) was calculated based on the ratio of the molar masses of GST (25.0 kDa) to VP1 (42.5 kDa), i.e., 1 mgL⁻¹ of GST-VP1 contains 42.5/67.5=0.6 mgL⁻¹ of VP1. The response variable Y_{S} was transformed with a square-root function as determined by the Box-Cox method. Significance of each factor investigated was assessed with a half-normal probability plot and checked with analysis of variance (ANOVA).

Expression of VP1 Protein

Glycerol stocks of transformed cells were streaked onto an LB agar plate (15 gL⁻¹ agar, 10 gL⁻¹ peptone, 5 gL⁻¹ yeast extract, 10 gL⁻¹ NaCl, pH 7.0), from which single colonies were obtained for seed cultures. 50 mL TB medium [12 gL⁻¹ peptone, 24 gL⁻¹ yeast extract, 0.4% (v/v) glycerol, 2.31 gL⁻¹ KH₂PO₄ and 12.54 gL⁻¹ K₂HPO₄] was inoculated with seed culture and cultivated on a rotary shaker (180 rpm, Ratek, VIC, Australia) at 26° C. or 37° C. Cultures were induced with IPTG when the cell OD₆₀₀ reached 0.5 or 4.0 (±10%) and were then grown for a further 6 h at 26° C., after which cell pellets were collected by centrifugation (22,000 g, 20 min, 4° C.). Cultures cultivated at 37° C. before induction were cooled with running tap water at room temperature for 1 min immediately before IPTG addition. All cultures were supplemented with 100 mgL⁻¹ ampicillin, and additionally 34 mgL⁻¹ chloramphenicol for RosDS cultures.

Bioanalyzer Analysis

Cell pellets were resuspended in L Buffer [40 mM Tris (pH 8.0), 200 mM NaCl, 1 mM EDTA, 5% (v/v) glycerol, 5 mM DTT] (Chen et al., 2001), sonicated with a Branson Sonifier 250 cell disruptor mounted with a microtip (Branson Ultrasonics Corporation, CT, USA) for three 10 s bursts (30 W) and centrifuged (22000 g, 15 min, 4° C.). The supernatant was analyzed in randomized order using a 2100 Bioanalyzer™ (Agilent Technologies, CA, USA) with Protein 200 or 230 LabChip kits according to the manufacturer's protocol.

Purification and Assembly of VP1

To prepare protein sufficient for subsequent purification and assembly experiment into virus-like particles, RosDS-pGEXVP1 and RosDS-pGEXVP1COpt cells were cultivated in 400 mL TB medium at 37° C. and cooled to 26° C. when cell OD₆₀₀ was 4.0 (±10%). The cells were induced with 1 mM IPTG and harvested by centrifugation (6900 g, 20 min, 4° C.) 6 h after induction. Two cell pellets, each from a 400 mL culture, were resuspended in 120 mL of ice-cold L Buffer and homogenized (Niro-Soavi, Parma, Italy) at 1000 bar. The homogenate was clarified by centrifugation (2000 g, 30 min, 4° C.) and filtration through 0.22 μm filters (Pall, New York, USA). GST-VP1 was captured with a 5 mL GSTrap HP column (GE Healthcare Biosciences) and eluted with E Buffer [40 mM Tris, pH 8.0, 10 mM reduced glutathione, 200 mM NaCl, 1 mM EDTA, 5% (v/v) glycerol, 5 mM DTT]. GST was cleaved from VP1 with 50 units per milliliter of thrombin (GE Healthcare Biosciences), at room temperature for 2 h, and removed with a Superdex 200 30/100 GL column (GE Healthcare Biosciences) pre-equilibrated with L Buffer at 0.5 mLmin⁻¹. Purified VP1 capsomeres were assembled into virus-like particles (VLPs) by dialysis at 300 mgL⁻¹ into Assembly Buffer 1 [0.5 M (NH₄)₂SO₄, 20 mM Tris, pH 7.4, 5% (v/v) glycerol, 1 mM CaCl₂] for 15 h, and then against Assembly Buffer 2 [200 mM NaCl, 20 mM Tris, pH 7.4, 5% (v/v) glycerol, 1 mM CaCl₂] for another 24 h (Gleiter and Lilie, 2001).

Transmission Electron Microscopy

Two microlitres of assembled VLPs at 200 mgL⁻¹ was applied to glow-discharged, 200-mesh carbon-coated grids (Proscitech, QLD, Australia). Remaining liquid on the grids was blotted with filter paper after 2 min. Grids were washed with water, stained with 2% (w/v) uranyl acetate for 30 s, dried and visualized with a Jeol 1011 (Jeol Ltd., Tokyo, Japan) microscope at 100 kV. Electron micrographs were recorded digitally using a side-mounted Morada camera (Olympus-Soft Imaging System GmbH, Münster, Germany) with iTEM software (version 3.2, Soft Imaging System GmbH).

Expression Optimization by Design of Experiment

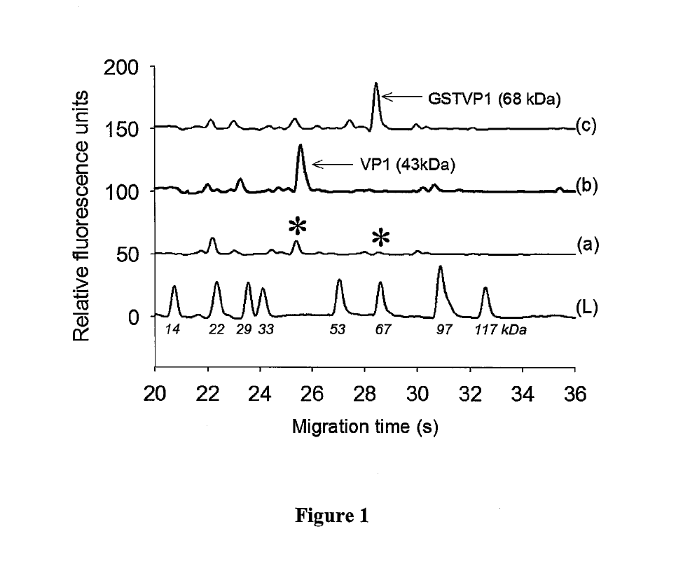

The viral structural protein VP1 from murine polyomavirus was originally expressed in RB791 cells by the ptacVP1 vector with a poor yield (2-3% of total protein) (Leavitt et al., 1985) that has not seen substantial improvements in later studies (Schmidt et al., 2000, Braun et al., 1999). We investigated in our factorial experiment the possibility of yield improvement by using sophisticatedly engineered host strains such as RosDS and expression vector (pGEX) that couples the expressed protein with a highly soluble fusion partner such as glutathione-S-transferase (GST). We also varied several cultivation conditions that are known to influence protein expression in E. coli, such as induction cell density, IPTG concentration, and pre-induction temperature (Hockney, 1994, Georgiou and Valax, 1996, Makrides, 1996, Hannig and Makrides, 1998, Sorensen and Mortensen, 2005, Jaganaman et al., 2007). Table 1 reports the normalized expression level of soluble VP1 (Y_{S}) for the 32 conditions investigated. The best expression level was 23 mgL⁻¹OD⁻¹ obtained in Run 16 (C=0.5, D=1.00 mM, E=26° C.). The electropherogram for this run [FIG. 1, plot (b)] shows a dominant peak corresponding to approximately 43 kDa, among other minor peaks of 22, 29 and 97 kDa that are presumably host proteins. In fact, host proteins of approximately 68 and 43 kDa were also detected in untransformed cells [FIG. 1, plot (a)], and treated as background signals which had been subtracted from the measured response for GST-VP1 and VP1 in induced cells. The subtraction subsequently yielded negative response value when protein expression was insignificant compared to the background (e.g. Runs 1, 3, 15 and 32 in Table 1). FIG. 1 [plot (c)] shows that GST-VP1 was highly expressed in Run 12 (C=4.0, D=1.00 mM, E=26° C.), giving a VP1 yield of 11 mgL⁻¹ OD⁻¹. The expression of GST-VP1 was of interest because use of the GST fusion tag can potentially simplify downstream purification, improving overall yield and purity of VP1.

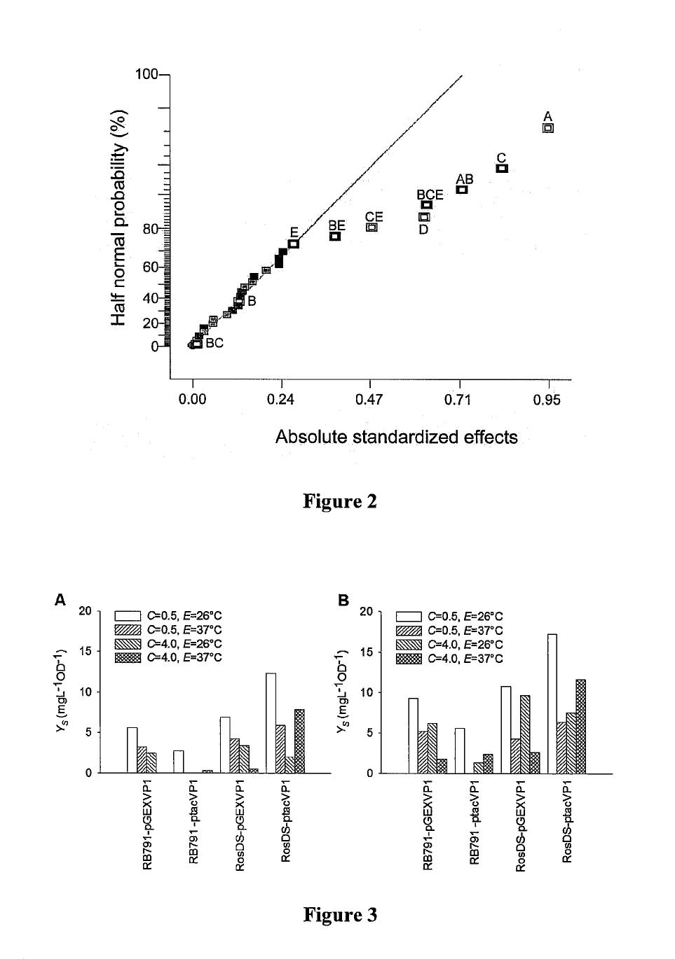

FIG. 2 shows the half-normal probability plot for isolation of significant effects. Two clusters of data were clearly observable: significant effects that lie close to the right side of the graph and near-zero effects which form a line at the far left corner of the graph (Montgomery, 2005). The important effects (including interactions) identified are A, C, AB, BC, BE, CE, and BCE. Other effects such as B and E, although not significant, were also included to support the model hierarchy for internal consistency in the model (Montgomery, 2005). The resulting model for the expression level of soluble VP1 is:

\sqrt{Yₛ+1.40}=2.34+0.47A+0.06B−0.41C+0.31D−0.14E−0.36AB+0.01BC−0.19BE+0.24CE−0.31BCE (1)

Table 2 presents analysis of variance (ANOVA) of the model, highlighting the significant model terms A, C, D, AB, BE, CE and BCE as predicted by the half-normal probability plot. The lack-of-fit test result, as depicted by the model p-value (<0.0001), indicates that the model is highly significant. An R-squared value of 0.90 also reflects the reliability of the model to predict experimental outcome. This was also confirmed by a calculated value of 16.04 for the “adequate precision” term, which satisfied the minimum requirement for an accurate model (>4) (Montgomery, 2005). FIG. 3 clearly shows that the host strain RosDS was superior to RB791 for all conditions tested. In addition, the expression of soluble VP1 was generally enhanced by induction at a lower cell density with a higher IPTG concentration, except for a few instances. One of the major advantages of using statistical analysis is that the interactions among multiple factors can be readily determined, as demonstrated by equation 1. The significance of the host-plasmid interaction (AB) was seen when the cells were pre-cultivated at 26° C. before induction at an OD₆₀₀ of 0.5. Under these conditions pGEXVP1 gave a greater yield than ptacVP1 in RB791, whereas the effect was opposite in RosDS. In addition, higher induction OD₆₀₀ appeared to be detrimental for the expression of VP1 if the cultures were pre-cultivated at 26° C. before induction, but increased the yield of VP1 in RosDS-ptacVP1 when a pre-induction temperature of 37° C. was used. This result highlights the importance of the interaction between induction OD₆₀₀ and pre-induction temperature (CE).

Codon Optimization of VP1 Gene

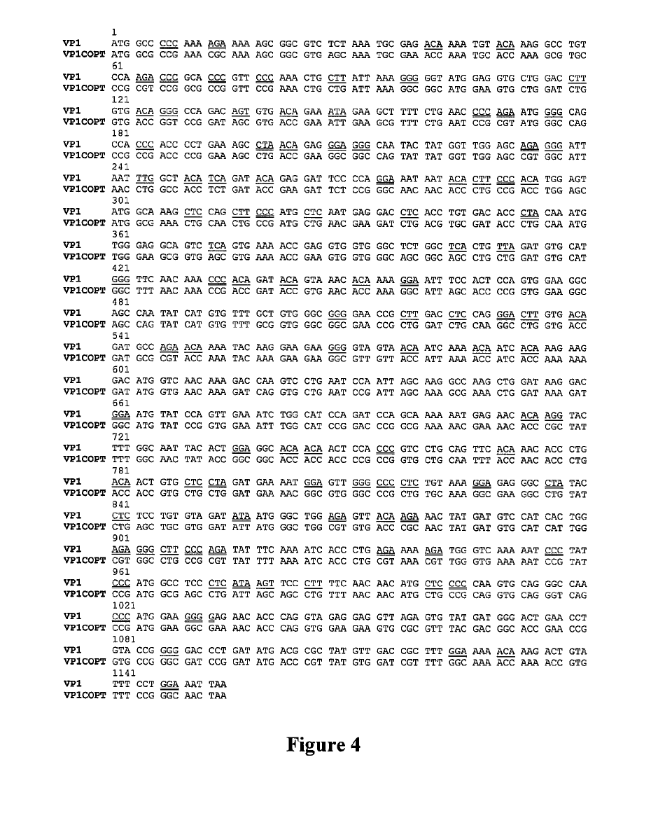

Enhanced expression of soluble VP1 in RosDS cells suggests the importance of codon usage. The wild-type VP1 gene contains 123 out of 385 (32%) codons which are considered rare to E. coli (underlined in FIG. 4). This high content of rare codons may have led to translational stalling due to the depletion of the corresponding tRNAs, resulting in a slow rate of protein expression and rendering the mRNAs susceptible to degradation (Varenne and Lazdunski, 1986, Makrides, 1996, Sorensen and Mortensen, 2005). Rare codon usage was reported to negatively affect the expression of viral structural proteins, including bovine papillomavirus L1 (Zhou et al., 1999) and enterovirus VP1 (Chen et al., 2004). The latter study also suggested that the arrangement of the existing rare codons may play another important role in suppressing expression of the target gene. Problems caused by rare codon usage can potentially be resolved through site-directed mutagenesis of the target gene, substituting the rare codons with ones that better mirror the availability of tRNAs in E. coli (Sorensen and Mortensen, 2005, Hannig and Makrides, 1998). Another more time- and cost-effective alternative is to co-express the genes encoding the tRNA cognate to the rare codons in the host systems, overcoming the need to optimize each gene sequence. E. coli cell strains which harbor these genes such as BL21(DE3)CodonPlus-RIL, BL21(DE3)CodonPlus-RP (Stratagene, Calif., USA) and Rosetta(DE3)pLysS (Novagen) are commercially available. Choi et al (Choi et al., 2007) reported yield enhancement of 5-10 times when Rosetta(DE3)pLacI was used to express rotavirus VP6, but the use of Rosetta 2(DE3) or Rosetta (DE3)pLysS failed to improve the expression of VP1 from enterovirus type 70 (Chen et al., 2004). Our results suggest the likelihood of correlation between VP1 expression level and E. coli rare codon usage of the encoding gene, although the lack of ompT and Ion proteases in RosDS may have also reduced the chance of in vivo proteolytic degradation, resulting in a higher overall VP1 expression. The pLysSRARE plasmid of Rosetta(DE3)pLysS strain used in this study encodes the tRNAs for the rare codons ATA, AGG, AGA, CTA, CCC, GGA (double-underlined in FIG. 4), thereby reducing the percentage of effective E. coli rare codons in VP1 gene from 32% to 20%. It is possible that elimination of these “remaining” rare codons may further improve VP1 expression in RosDS. Furthermore, purity of the expressed protein may be enhanced if products resulting from translational stalling can be reduced by codon optimization.

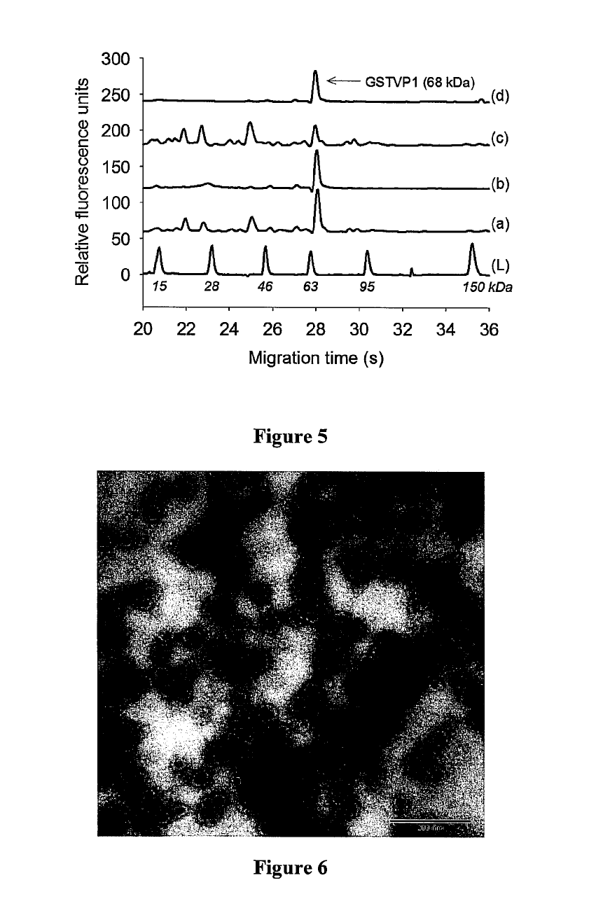

To further investigate the rare codon effect, we created the pGEXVP1COpt vector containing a redesigned VP1 gene that is deficient of E. coli rare codons (FIG. 4). In addition, regions of very high (>80%) or very low (<30%) GC content were substituted, where possible. We compare expression of VP1 from wild-type and codon-optimized genes at conditions previously optimized for RosDS-pGEXVP1. FIG. 5 shows that full codon optimization did not further increase VP1 expression in RosDS. We also tested GST-VP1 purified by affinity chromatography after expression by pGEXVP1COpt [FIG. 5, plots (b) and (d)] and observed no significant improvement in quality of the purified protein. Despite many studies reporting successes of codon optimization (Pikaart and Felsenfeld, 1996, Hale and Thompson, 1998, Kleber-Janke and Becker, 2000, Nishikubo et al., 2005, Yazdani et al., 2006), there were exceptions where this approach was shown to be ineffective (Alexeyev and Winkler, 1999, Griswold et al., 2003, Wu et al., 2004), potentially due to reasons related to “over-optimization” (Gustafsson et al., 2004, Wu et al., 2006), including (i) imbalanced tRNA pool caused by strongly transcribed mRNAs that leads to translational error; (ii) inhibition of ribosome processivity due to repetitive elements and secondary structures in the gene and mRNA introduced during codon optimization, and (iii) elimination of non-optimal codons which are important for folding of nascent translated polypeptide. Codon optimization performed with GeneOptimizer™ (Geneart AG) should nonetheless eliminate any possible RNA secondary structure according to the software manufacturer. Our results indicated that full codon optimization is unnecessary for enhancement of VP1 expression; instead, a simpler and cheaper alternative using a strategically modified host cell such as RosDS provided formidable expression improvement (FIG. 3) for the rare-codon rich gene.

Within the scope of our investigation, the best condition to express murine polyomavirus VP1 was with RosDS-ptacVP1 at a pre-induction temperature of 26° C. and an induction OD₆₀₀ of 0.5. Under these conditions, the expression yield of soluble VP1 was approximately 180 mg per L of culture harvested at late log phase (OD₆₀₀=8). This result represents at least an order-of-magnitude improvement over yields previously reported (Leavitt et al., 1985, Braun et al., 1999, Schmidt et al., 2000). The purification of un-tagged VP1 can be very time-consuming and inefficient (Leavitt et al., 1985), resulting in very low overall recovery of VP1. Alternatively, VP1 can be expressed as a GST fusion protein by using RosDS-pGEXVP1 at the same pre-induction temperature but at a higher induction OD₆₀₀ of 4.0, giving a lower expression of 90 mg per L of culture (OD₆₀₀=8) that is compensated by the ease and efficiency of purification with GST affinity chromatography. Based on these findings, it is envisaged that grams-per-liter expression levels of viral structural protein are achievable in high-cell-density cultures (OD₆₀₀>100) of E. coli. We further demonstrated that the purified VP1 capsomeres, after release from GST, are functional, and can be further processed and assembled into virus-like particles under a physicochemically controlled environment (FIG. 6).

TABLE 1
Experimental design matrix and response for
expression of soluble VP1 in E. coli.

| | | | Factors^{#} | | | | | |
|---|---|---|---|---|---|---|---|---|
| | Standard | | | | | | | Response: Yₛ |
| | Order* | Run | A | B | C | D | E | (mgL⁻¹ OD⁻¹) |
| BLOCK 1 | 25 | 1 | RB791 | ptacVP1 | 0.5 | 1.00 | 37 | −0.44 |
| | 31 | 2 | RB791 | pGEXVP1 | 4.0 | 1.00 | 37 | 1.55 |
| | 1 | 3 | RB791 | ptacVP1 | 0.5 | 0.05 | 26 | −0.01 |
| | 7 | 4 | RB791 | pGEXVP1 | 4.0 | 0.05 | 26 | 0.59 |
| | 20 | 5 | RosDS | pGEXVP1 | 0.5 | 0.05 | 37 | 3.95 |
| | 14 | 6 | RosDS | ptacVP1 | 4.0 | 1.00 | 26 | 2.56 |
| | 22 | 7 | RosDS | ptacVP1 | 4.0 | 0.05 | 37 | 12.66 |
| | 12 | 8 | RosDS | pGEXVP1 | 0.5 | 1.00 | 26 | 6.83 |
| BLOCK 2 | 27 | 9 | RB791 | pGEXVP1 | 0.5 | 1.00 | 37 | 3.45 |
| | 3 | 10 | RB791 | pGEXVP1 | 0.5 | 0.05 | 26 | 5.42 |
| | 29 | 11 | RB791 | ptacVP1 | 4.0 | 1.00 | 37 | 2.06 |
| | 16 | 12 | RosDS | pGEXVP1 | 4.0 | 1.00 | 26 | 11.42 |
| | 18 | 13 | RosDS | ptacVP1 | 0.5 | 0.05 | 37 | 5.29 |
| | 24 | 14 | RosDS | pGEXVP1 | 4.0 | 0.05 | 37 | 0.67 |
| | 5 | 15 | RB791 | ptacVP1 | 4.0 | 0.05 | 26 | −2.07 |
| | 10 | 16 | RosDS | ptacVP1 | 0.5 | 1.00 | 26 | 23.08 |
| BLOCK 3 | 9 | 17 | RB791 | ptacVP1 | 0.5 | 1.00 | 26 | 5.68 |
| | 17 | 18 | RB791 | ptacVP1 | 0.5 | 0.05 | 37 | 0.72 |
| | 6 | 19 | RosDS | ptacVP1 | 4.0 | 0.05 | 26 | 4.38 |
| | 4 | 20 | RosDS | pGEXVP1 | 0.5 | 0.05 | 26 | 8.79 |
| | 30 | 21 | RosDS | ptacVP1 | 4.0 | 1.00 | 37 | 17.55 |
| | 28 | 22 | RosDS | pGEXVP1 | 0.5 | 1.00 | 37 | 9.61 |
| | 15 | 23 | RB791 | pGEXVP1 | 4.0 | 1.00 | 26 | 5.88 |
| | 23 | 24 | RB791 | pGEXVP1 | 4.0 | 0.05 | 37 | 1.39 |
| BLOCK 4 | 19 | 25 | RB791 | pGEXVP1 | 0.5 | 0.05 | 37 | 4.04 |
| | 2 | 26 | RosDS | ptacVP1 | 0.5 | 0.05 | 26 | 10.38 |
| | 11 | 27 | RB791 | pGEXVP1 | 0.5 | 1.00 | 26 | 11.41 |
| | 26 | 28 | RosDS | ptacVP1 | 0.5 | 1.00 | 37 | 8.00 |
| | 8 | 29 | RosDS | pGEXVP1 | 4.0 | 0.05 | 26 | 4.42 |
| | 32 | 30 | RosDS | pGEXVP1 | 4.0 | 1.00 | 37 | 0.71 |
| | 21 | 31 | RB791 | ptacVP1 | 4.0 | 0.05 | 37 | 6.36 |
| | 13 | 32 | RB791 | ptacVP1 | 4.0 | 1.00 | 26 | −2.29 |

*Standard order is the conventional order of the design matrix. After assigned to the appropriate blocks, the experiments were randomized and performed according to the run sequence.
^{#}Factors: (A) Host strain; (B) expression vector; (C) induction OD₆₀₀; (D) IPTG concentration in mM, and (E) pre-induction temperature in ° C.

TABLE 2
Analysis of variance (ANOVA) for model of soluble
VP1 expression in Escherichia coli.

| | Coefficient | | |
|---|---|---|---|
| Factor or interaction | estimate | p-Value | F-Value |
| A (Host strain)** | 0.47 | <0.0001 | 45.79 |
| B (Plasmid) | 0.06 | 0.3857 | 0.79 |
| C (Cell density at induction)** | −0.41 | 0.0001 | 34.70 |
| D (IPTG concentration)** | 0.31 | 0.0003 | 19.46 |
| E (pre-induction temperature) | −0.14 | 0.0695 | 3.73 |
| AB** | −0.36 | <0.0001 | 26.32 |
| BC | 0.01 | 0.9394 | 0.01 |
| BE* | −0.19 | 0.0140 | 7.40 |
| CE** | 0.24 | 0.0031 | 11.68 |
| BCE** | −0.31 | 0.0003 | 19.88 |

*Significant (p-value < 0.05);
**Highly significant (p-value < 0.01);
Model statistics: p-value < 0.0001;
F-value = 16.97;
R-squared = 0.90;
Adjusted R-squared = 0.85;
Adequate precision: 16.04;
Coefficient of variance: 16.95%.

### REFERENCES

ALEXEYEV, M. F. & WINKLER, H. H. (1999) Gene synthesis, bacterial expression and purification of the Rickettsia prowazekii ATP/ADP translocase. Biochimica Et Biophysica Acta-Biomembranes, 1419, 299-306.
BANEYX, F. (1999) Recombinant protein expression in Escherichia coli. Curr. Opin. Biotechnol., 10, 411-421.
BRAUN, H., BOLLER, K., LOWER, J., BERTLING, W. M. & ZIMMER, A. (1999) Oligonucleotide and plasmid DNA packaging into polyoma VP1 virus-like particles expressed in Escherichia coli. Biotechnol. Appl. Biochem., 29, 31-43.
CHEN, D. Q., DUGGAN, C., GANLEY, J. P., KOORAGAYALA, L. M., REDEN, T. B., TEXADA, D. E. & LANGFORD, M. P. (2004) Expression of enterovirus 70 capsid protein VP1 in Escherichia coli. Protein Expr. Purif., 37, 426-433.
CHEN, X. J. S., CASINI, G., HARRISON, S. C. & GARCEA, R. L. (2001) Papillomavirus capsid protein expression in Escherichia coli: Purification and assembly of HPV11 and HPV16 L1. J. Mol. Biol., 307, 173-182.
CHOI, A. H. C., BASU, M., MCNEAL, M. M., BEAN, J. A., CLEMENTS, J. D. & WARD, R. L. (2007) Intranasal administration of an Escherichia coli-expressed codon-optimized rotavirus VP6 protein induces protection in mice Protein Expr. Purif., 53, 238-238.
CHOI, J. H., KEUM, K. C. & LEE, S. Y. (2006) Production of recombinant proteins by high cell density culture of Escherichia coli. Chemical Engineering Science, 61, 876-885.
COOK, J. C., JOYCE, J. G., GEORGE, H. A., SCHULTZ, L. D., HURNI, W. M., JANSEN, K. U., HEPLER, R. W., IP, C., LOWE, R. S., KELLER, P. M. & LEHMAN, E. D. (1999) Purification of virus-like particles of recombinant human papillomavirus type 11 major capsid protein L1 from Saccharomyces cerevisiae. Protein Expr. Purif., 17, 477-484.
GEORGIOU, G. & VALAX, P. (1996) Expression of correctly folded proteins in Escherichia coli. Curr. Opin. Biotechnol., 7, 190-197.
GLEITER, S. & LILIE, H. (2001) Coupling of antibodies via protein Z on modified polyoma virus-like particles. Protein Sci., 10, 434-444.
GRISWOLD, K. E., MAHMOOD, N. A., IVERSON, B. L. & GEORGIOU, G. (2003) Effects of codon usage versus putative 5′-mRNA structure on the expression of Fusarium solani cutinase in the Escherichia coli cytoplasm. Protein Expr. Purif., 27, 134-142.
GUSTAFSSON, C., GOVINDARAJAN, S. & MINSHULL, J. (2004) Codon bias and heterologous protein expression. Trends Biotechnol., 22, 346-353.
HALE, R. S. & THOMPSON, G. (1998) Codon optimization of the gene encoding a domain from human type 1 neurofibromin protein results in a threefold improvement in expression level in Escherichia coli. Protein Expr. Purif., 12, 185-188.
HANNIG, G. & MAKRIDES, S. C. (1998) Strategies for optimizing heterologous protein expression in Escherichia coli. Trends Biotechnol., 16, 54-60.
HENAUT, A. & DANCHIN, A. (1996) Analysis and predictions from Escherichia coli sequences. IN NEIDHARDT, F. C. & CURTISS, R. (Eds.) Escherichia coli and Salmonella: cellular and molecular biology 2nd ed. Washington, D. C., ASM Press.
HOCKNEY, R. C. (1994) Recent developments in heterologous protein production in Escherichia coli. Trends Biotechnol., 12, 456-463.
JAGANAMAN, S., PINTO, A., TARASEV, M. & BALLOU, D. P. (2007) High levels of expression of the iron-sulfur proteins phthalate dioxygenase and phthalate dioxygenase reductase in Escherichia coli. Protein Expr. Purif., 52, 273-279.
KLEBER-JANKE, T. & BECKER, W. M. (2000) Use of modified BL21(DE3) Escherichia coli cells for high-level expression of recombinant peanut allergens affected by poor codon usage. Protein Expr. Purif., 19, 419-424.
KOUTSKY, L. A., AULT, K. A., WHEELER, C. M., BROWN, D. R., BARR, E., ALVAREZ, F. B., CHIACCHIERINI, L. M., JANSEN, K. U. & INVESTIGATO, P. P. S. (2002) A controlled trial of a human papillomavirus type 16 vaccine. N. Engl. J. Med., 347, 1645-1651.
LEAVITT, A. D., ROBERTS, T. M. & GARCEA, R. L. (1985) Polyoma-Virus Major Capsid Protein, Vp1—Purification after High-Level Expression in Escherichia coli. J. Biol. Chem., 260, 2803-2809.
MAKRIDES, S. C. (1996) Strategies for achieving high-level expression of genes in Escherichia coli. Microbiological Reviews, 60, 512-&.
MONTGOMERY, D. C. (2005) Design and analysis of experiments, Hoboken, NJ John Wiley & Sons.
NIKEREL, I. E., TOKSOY, E., KIRDAR, B. & YILDIRIM, R. (2005) Optimizing medium composition for TaqI endonuclease production by recombinant Escherichia coli cells using response surface methodology. Process Biochem., 40, 1633-1639.
NISHIKUBO, T., NAKAGAWA, N., KURAMITSU, S. & MASUI, R. (2005) Improved heterologous gene expression in Escherichia coli by optimization of the AT-content of codons immediately downstream of the initiation codon. J. Biotechnol., 120, 341-346.
PATTENDEN, L. K., MIDDELBERG, A. P. J., NIEBERT, M. & LIPIN, D. I. (2005) Towards the preparative and large-scale precision manufacture of virus-like particles. Trends Biotechnol., 23, 523-529.
PIKAART, M. J. & FELSENFELD, G. (1996) Expression and codon usage optimization of the erythroid-specific transcription factor cGATA-1 in baculoviral and bacterial systems. Protein Expr. Purif., 8, 469-475.
PUSHKO, P., TUMPEY, T. M., BU, F., KNELL, J., ROBINSON, R. & SMITH, G. (2005) Influenza virus-like particles comprised of the HA, NA, and M1 proteins of H9N2 influenza virus induce protective immune responses in BALB/c mice. Vaccine, 23, 5751-5759.
PUSHKO, P., TUMPEY, T. M., HOEVEN, N. V., BELSER, J. A., ROBINSON, R., NATHAN, M., SMITH, G., WRIGHT, D. C. & BRIGHT, R. A. (2007) Evaluation of influenza virus-like particles and Novasome adjuvant as candidate vaccine for avian influenza. Vaccine, 25, 4283-4290.
RAYMENT, I., BAKER, T. S., CASPAR, D. L. D. & MURAKAMI, W. T. (1982) The structure of polyoma-virus capsids at 22.5-a resolution. Biophys. J., 37, A8-A8.
SALUNKE, D. M., CASPAR, D. L. D. & GARCEA, R. L. (1986) Self-assembly of purified polyomavirus capsid protein VP1. Cell, 46, 895-904.
SAMBROOK, J. & RUSSELL, D. W. (2001) Molecular cloning: a laboratory manual, Cold

Spring Harbor, N.Y., Cold Spring Harbor Laboratory Press.
SCHMIDT, U., RUDOLPH, R. & BOHM, G. (2000) Mechanism of assembly of recombinant murine polyomavirus-like particles. J. Virol., 74, 1658-1662.
SORENSEN, H. P. & MORTENSEN, K. K. (2005) Advanced genetic strategies for recombinant protein expression in Escherichia coli. J. Biotechnol., 115, 113-128.
SWALLEY, S. E., FULGHUM, J. R. & CHAMBERS, S. P. (2006) Screening factors effecting a response in soluble protein expression: Formalized approach using design of experiments. Anal. Biochem., 351, 122-127.
VARENNE, S. & LAZDUNSKI, C. (1986) Effect of distribution of unfavorable codons in the maximum rate of gene-expression by an heterologous organism. J. Theor. Biol., 120, 99-110.
VILLA, L. L., COSTA, R. L. R., PETTA, C. A., ANDRADE, R. P., PAAVONEN, J., IVERSEN, O. E., OLSSON, S. E., HOYE, J., STEINWALL, M., RIIS-JOHANNESSEN, G., ANDERSSON-ELLSTROM, A., ELFGREN, K., VON KROGH, G., LEHTINEN, M., MALM, C., TAMMS, G. M., GIACOLETTI, K., LUPINACCI, L., RAILKAR, R., TADDEO, F. J., BRYAN, J., ESSER, M. T., SINGS, H. L., SAAH, A. J. & BARR, E. (2006) High sustained efficacy of a prophylactic quadrivalent human papillomavirus types 6/11/16/18 L1 virus-like particle vaccine through 5 years of follow-up. Br. J. Cancer, 95, 1459-1466.
WANG, Y. H., JING, C. F., YANG, B., MAINDA, G., DONG, M. L. & XU, A. L. (2005) Production of a new sea anemone neurotoxin by recombinant Escherichia coli: Optimization of culture conditions using response surface methodology. Process Biochem., 40, 2721-2728.
WROBEL, B., YOSEF, Y., OPPENHEIM, A. B. & OPPENHEIM, A. (2000) Production and purification of SV40 major capsid protein (VP1) in Escherichia coli strains deficient for the GroELS chaperone machine. J. Biotechnol., 84, 285-289.
WU, G., BASHIR-BELLO, N. & FREELAND, S. J. (2006) The Synthetic Gene Designer: A flexible web platform to explore sequence manipulation for heterologous expression. Protein Expr. Purif., 47, 441-445.
WU, X. Q., JORNVALL, H., BERNDT, K. D. & OPPERMANN, U. (2004) Codon optimization reveals critical factors for high level expression of two rare codon genes in Escherichia coli: RNA stability and secondary structure but not tRNA abundance. Biochem. Biophys. Res. Commun., 313, 89-96.
YAZDANI, S. S., SHAKRI, A. R., PATTNAIK, P., RIZVI, M. M. A. & CHITNIS, C. E. (2006) Improvement in yield and purity of a recombinant malaria vaccine candidate based on the receptor-binding domain of Plasmodium vivax Duffy binding protein by codon optimization. Biotechnol. Lett., 28, 1109-1114.
ZHOU, J., LIU, W. J., PENG, S. W., SUN, X. Y. & FRAZER, I. (1999) Papillomavirus capsid protein expression level depends on the match between codon usage and tRNA availability. J. Virol., 73, 4972-4982.
   Each publication cited herein is incorporated by reference in its entirety.

While various embodiments of the present invention have been described in detail herein, it is apparent that modifications and adaptations of those embodiments will occur to those skilled in the art. It is to be expressly understood, however, that such modifications and adaptations are within the scope of the present invention, as set forth in the following exemplary claims:

We claim:

1. A method for producing a protein comprising:
a) culturing bacteria until the bacteria reach stationary phase, wherein the bacteria express a recombinant nucleic acid molecule encoding the protein and wherein expression of the protein is inducible;
b) cooling the culture to about 25° C.;
c) inducing protein expression by adding an induction agent; and
d) culturing the bacteria at about 25° C.

2. The method of claim 1, further comprising a step of recovering the protein from the bacteria or the culture.

3. The method of claim 2, wherein the step of recovering the protein comprises purifying the protein.

4. The method of claim 3, wherein the step of recovering the protein comprises purifying the protein by chromatography.

5. The method of claim 3, wherein the step of recovering the protein comprises purifying the protein on a GST column.

6. The method of claim 1, wherein the protein is a fusion protein.

7. The method of claim 6, wherein the fusion protein is a GST fusion protein.

8. The method of claim 6, wherein the fusion protein is a viral protein fused to GST.

9. The method of claim 8, wherein the viral protein is a HPV or polyoma capsid protein.

10. The method of claim 8, wherein the fusion protein is GST-HPV L1 or GST-HPV L2.

11. The method of claim 9, wherein the viral protein is a polyoma VP1 protein.

12. The method of claim 1, wherein the bacteria are transfected with the recombinant nucleic acid molecule less than about 48 hours prior to step (a).

13. The method of claim 1, wherein step (a) comprises growing a starter culture of the bacteria.

14. The method of claim 1, wherein the bacteria are E. coli.

15. The method of claim 1, wherein the bacteria are cultured in Terrific Broth.

16. The method of claim 1, wherein the recombinant nucleic acid molecule comprises a pGEX expression vector.

17. The method of claim 1, wherein the induction agent is IPTG.

18. The method of claim 1, wherein the bacteria are cultured in step (d) for at least about 4 hours.

19. A method for producing a GST-HPV L1 fusion protein or a MPV VP1 protein comprising:
a) culturing E. coli until the E. coli reach stationary phase, wherein the E. coli express a recombinant nucleic acid molecule encoding the GST-HPV L1 fusion protein or the MPV VP1 protein and wherein expression of the protein is inducible by IPTG;
b) cooling the culture to about 25° C.;
c) inducing protein expression by adding an IPTG; and
d) culturing the bacteria at about 25° C.

20. The method of claim 19, further comprising a step of recovering the protein from the E. coli or the culture.

21. The method of claim 20, wherein the step of recovering the GST-HPV L1 fusion protein comprises purifying the GST-HPV L1 fusion protein.

22. The method of claim 21, wherein the step of recovering the GST-HPV L1 fusion protein comprises purifying the GST-HPV L1 fusion protein by chromatography.

23. The method of claim 21, wherein the step of recovering the GST-HPV L1 fusion protein comprises purifying the GST-HPV L1 fusion protein on a GST column.

24. The method of claim 19, wherein the E. coli are transfected with the recombinant nucleic acid molecule less than about 48 hours prior to step (a).

25. The method of claim 19, wherein step (a) comprises growing a starter culture of the E. coli.

26. The method of claim 19, wherein the E. coli are cultured in Terrific Broth.

27. The method of claim 19, wherein the recombinant nucleic acid molecule comprises a pGEX expression vector.

28. The method of claim 19, wherein the bacteria are cultured in step (d) for at least about 4 hours.
